(19) United States
(12) Patent Application Publication
(10) Pub. No.: US 2015/0289841 A1
(43) Pub. Date: Oct. 15, 2015

(54) Portable Ultrasound Machine

(71) Applicant: ESAOTE SPA, Genova (IT)

(72) Inventors: Marco Brusacá, Genova (IT); Roberto Furia, Genova (IT)

(21) Appl. No.: 14/437,596

(22) PCT Filed: Sep. 27, 2013
(86) PCT No.: PCT/IB2013/058922

§ 371 Date: Apr. 22, 2015

(30) Foreign Application Priority Data
Oct. 26, 2012 (IT) GE2012A000103

(51) Int. Cl. A61B 8/00

(52) U.S. Cl. CPC A61B 8/4427; A61B 8/462; A61B 8/464; A61B 8/4433; A61B 8/4444

(57) ABSTRACT

A portable ultrasound machine, configured to be removably coupled to a supporting structure, includes a main body and a monitor connected to the main body by a joint. The main body has an upper surface with a control user interface, a lower surface resting in a housing seat of the supporting structure, and one or more side walls connecting the upper surface to the lower surface. The monitor has a front emitting side and a rear casing side. A communication interface between the machine and the supporting structure includes the joint, articulated such that the monitor is alternately movable in a first position parallel to the upper surface, with the front emitting side facing towards the upper surface, a second position substantially vertical above the upper surface, and a third position parallel to the lower surface, with the front emitting side facing towards the lower surface.

The present invention relates to a portable ultrasound machine, provided with means for the removable coupling to a supporting structure, comprising a main body and a monitor connected to said main body by means of a joint, which main body has an upper surface provided with a control user interface and a lower surface resting in a suitable housing seat of said supporting structure and one or more side walls connecting said upper surface to said lower surface, said monitor being provided with a front emitting side and a rear casing side opposite to said front emitting side, and there being provided a communication interface between said machine and said supporting structure.

Nowadays this type of portable ultrasound machines are used for combining portability advantages typical of a portable machine with the greater operability of a supporting structure, such as for example a carriage, connected with the machine itself, wherein peripheral devices or operating units enhancing the potentialities of the ultrasound machine can be housed.

Portable machines offer a great handiness but they are limited to the functional characteristics that result from the constructional elements that compose the machine itself, including the monitor, which usually has small dimensions, smaller than or comparable with the total dimensions of the ultrasound machine.

The connection of the portable machine to the supporting structure allows further peripheral devices and processing units and monitors fixed to the supporting structure to be used, which typically have dimensions greater than those of the monitor of the portable machine.

In known ultrasound machines, once they are mounted on the supporting structures, currently it is the monitor of the portable machine to be used, with a clear restriction as regards display possibilities; as an alternative, when the monitor of the supporting structure is present, both the monitors are kept visible, resulting in an increase in the total dimensions of the ultrasound system.

Therefore there is the unsatisfied need of managing the presence and the overall dimensions of the monitor of the portable machine and of the monitor of the supporting structure in relation to the desired conditions of use, and contemporaneously of guaranteeing a system as compact and functional as possible as a whole.

In some currently known devices said joint is articulatable such that said monitor is movable alternately in a first position where it is arranged parallel to said upper surface, with the front emitting side facing towards said upper surface and in a second position where it is arranged substantially vertical above said upper surface.

Documents WO2006/111871A1 and EP 2510880A1 describe portable ultrasound machines of such type, where however since the monitor is closed in a conventional manner by folding it on the upper surface such to become non-operating, it inhibits any possible operation of the user control interface.

This prevents the device from being used with the monitor in the non-operating condition, preferably due to an additional larger monitor of the supporting structure.

The invention aims at overcoming prior art-related drawbacks mentioned above by providing an ultrasound machine as described at the beginning, wherein in addition said joint is articulatable such that said monitor is alternately movable in a first position where it is arranged parallel to said upper surface, with the front emitting side facing towards said upper surface, in a second position where it is arranged substantially vertical above said upper surface and in a third position where it is arranged parallel to said lower surface, with the front emitting side facing towards said lower surface.

Therefore the present invention has the great advantage of allowing the monitor to be moved in several positions depending on the use of the machine.

In the first position the monitor is folded on the upper surface, as in a portable computer. This position can be taken for example when the machine is off or in stand-by and it allows the machine to be easily transported, since both the control interface and the front emitting side of the monitor are protected they being folded one on each other.

The second position, where the monitor is arranged substantially vertical above the upper surface, is the operating position that allows the user to display the acquired ultrasound images.

In the third position the monitor is folded against the lower interface, and therefore is it again in a non-operating position as in the first position. But unlike what occurs in the first position, in the third position the monitor does not cover the control interface, which therefore can be used by the user.

The monitor, in this third position, for example can be interposed between the ultrasound machine and the supporting structure when the machine is housed into said housing seat of the supporting structure.

In this case it is possible to use the machine as well, without using the monitor of the machine, which monitor is therefore advantageously concealed.

The lower and upper surfaces are to be intended with reference to a normal operating position, wherein the machine is used with the two surfaces substantially parallel to the horizontal plane, the upper surface provided with said control interface for the user is faced towards the user for a comfortable use.

Deviations from this position, for example directed to better ergonomics, however have to be intended to fall within the scope of the claims.

Similarly when the monitor takes said second position it can be in the vertical position or it can be optimally tilted in order to be seen by the user.

In one embodiment said joint has at least two pivot points, the axes of rotation of at least two of said pivot points being perpendicular to each other.

According to this embodiment a hinge allows the monitor to be brought from a substantially horizontal position to a substantially vertical position, while a pivot point allows the monitor to be rotated from a position where it is arranged vertical above the upper surface to a position where it is arranged vertical underneath the lower surface.

In a further example said joint has at least three pivot points, the axes of rotation of at least a first and at least a second pivot points being parallel to each other and perpendicular with respect to the axis of rotation of at least a third pivot point.

In a preferred embodiment the joint is composed of two portions hinged to each other by a medial hinge, a first portion of which is connected to a terminal rotationally coupling with the main body, and a second portion is connected to said monitor by an end hinge. The medial hinge and the end hinge have axes of rotation parallel to each other, while said rotational coupling terminal has an axis of rotation perpendicular to the other two.

This allows the monitor to be moved away from the main body by rotating the medial hinge, before rotating the joint about of the axis of rotation of the coupling terminal, and therefore it allows an easier rotation.

In a further embodiment said main body has a parallelepiped shape, such that said side walls are composed of a front wall, a rear wall and two side walls, said joint being connected to said rear wall at a point equidistant from said upper wall and from said lower wall.

Thus the monitor can be moved from said first to said third position by simple rotations of the articulatable joint, without the need of elongating said joint, such positions being symmetric with respect to a horizontal plane substantially passing by the centre of said main body.

According to a further embodiment there are provided means for coupling the machine to said supporting structure, which coupling means are positioned on said lower surface and/or on said side walls, on opposite sides of said lower surface, and they are spaced apart from each other to an extent corresponding to or greater than the dimensions of said monitor, such that when said monitor is moved in said third position it is interposed between said coupling means.

Such a positioning of the coupling means guarantees the possibility of moving the monitor in the third position and therefore of coupling the portable machine to the supporting structure, without the monitor, interposed between the portable machine and supporting structure, interfering with the coupling means.

In a further embodiment said supporting structure is provided with at least one additional monitor, said communication interface between said machine and said supporting structure being intended to display the images acquired by said machine on said additional monitor.

This allows a plurality of operating configurations to be provided depending on different needs, for example it is possible to couple the portable machine to the supporting structure alternately with the monitor in the second position, that is arranged vertical above the upper surface, or with the monitor in the third position, folded underneath the ultrasound machine.

Thus it is possible to use contemporaneously the monitor of the portable machine and the additional monitor of the supporting structure, or it possible to use only the additional monitor of the supporting structure instead of the monitor of the portable machine.

According to a further embodiment said additional monitor is removably connectable to said supporting structure, or it is connected to or connectable to an arm removably connectable to said supporting structure.

Thus it is possible to remove or add the additional monitor depending on whether one desires or not to use the monitor of the portable machine.

In a further embodiment said additional monitor is composed of said monitor, said monitor being detachable from said machine and it being mechanically and electrically connectable to said supporting structure.

This allows not only the monitor to be positioned in the above mentioned positions when the portable machine is not coupled with the supporting structure, but it allows also the monitor of the portable machine coupled to the supporting structure to be used when the portable machine is housed into the supporting structure.

According to one embodiment said joint is provided with a coupling terminal intended to removably engage with a corresponding coupling terminal provided on said main body or on said supporting structure, both the terminals being provided with electrical contacts that are automatically connectable when said terminals are coupled to each other and which guarantee the electrical connections when rotating said joint. In a further example said communication interface comprises contacts of the so called docking station type, such that the connection is automatically established upon placing said machine into said housing seat.

According to a further embodiment there is provided at least one transport handle composed of a recess obtained on said upper surface, the mouth of said recess being coverable at least partially by a removable plug made of elastically pliable material having such dimensions that it acts as a wrist rest for the user.

In one embodiment the portable ultrasound machine comprises a probe having a plurality of electroacoustic transducers for emitting ultrasonic pulses into a body under examination and for receiving the echoes generated by said body under examination; means for generating emitting signals; means for processing receiving signals generated by the probe for generating images.

According to a further embodiment, in a side wall there is provided at least one connector for at least one ultrasound probe there being provided at least one case protecting said at least one connector, which case is connected to said side surface such that it is movable from an opening position to a closing position and it is shaped such to form in the closing position an housing space for at least one cable of said at least one probe and to form an outlet opening for said cable.

According to a further embodiment, one end of said case is hinged to said side surface such that said case is rotatable from said opening position to said closing position where it adheres against said side surface, said case having a concave shape and it being provided with different depths such that in the direction opposite to said side wall it has a widening at said at least one connector and a narrowing at said outlet opening for said cable.

Therefore the connectors are protected by said case, which however has minimum overall dimensions, it being shaped such that it easily houses the connectors and the cables and it lets all the cables to go out through a single opening.

The present invention relates also to a supporting carriage for a portable ultrasound machine comprising means for removably fastening said portable ultrasound machine and at least one terminal provided with a connector for the removable mechanical and electrical coupling of a monitor.

These and other characteristics and advantages of the present invention will be more clear from the following description of some embodiments shown in the annexed drawings wherein:

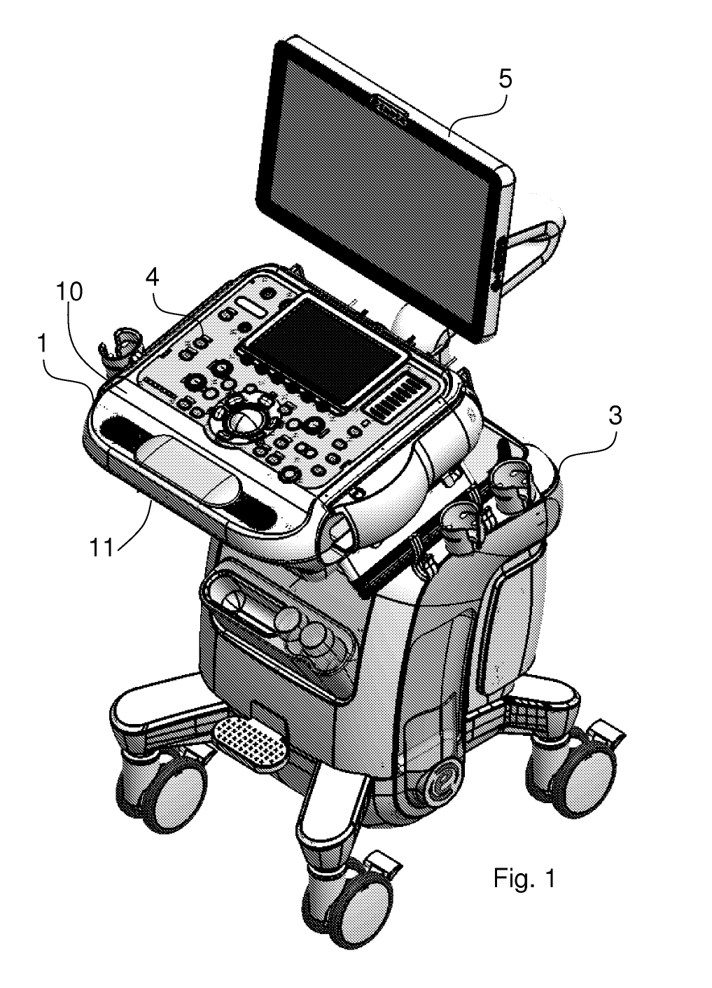

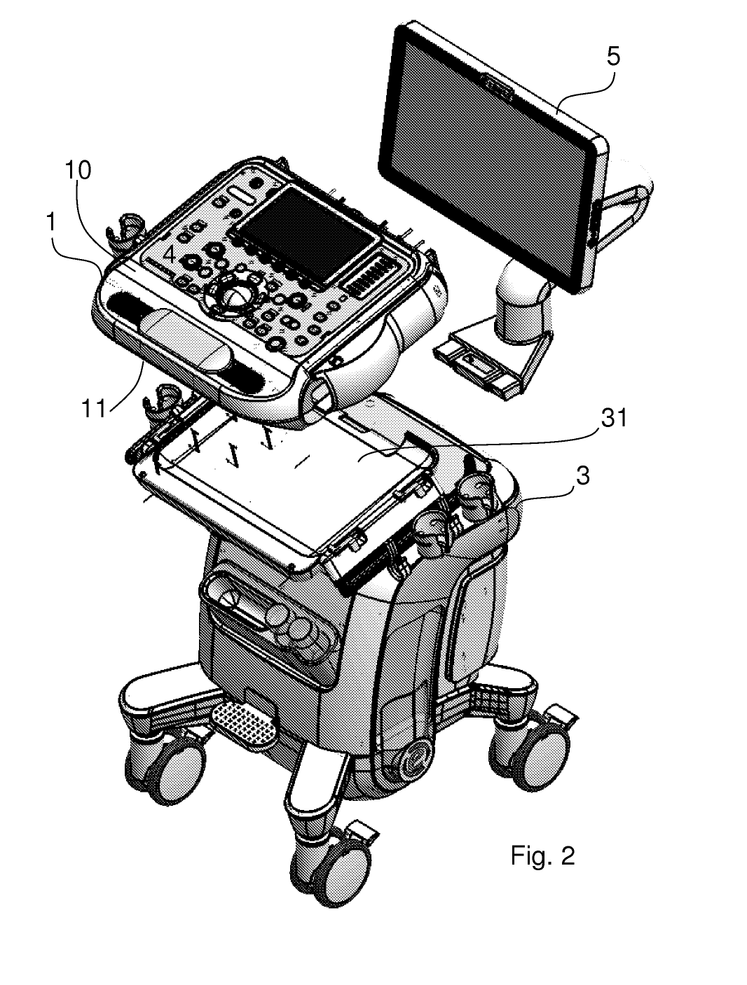

FIGS. 1 and 2 are one embodiment of the portable ultrasound machine of the present invention coupled with a supporting structure, particularly a carriage, and removed from the supporting structure respectively;

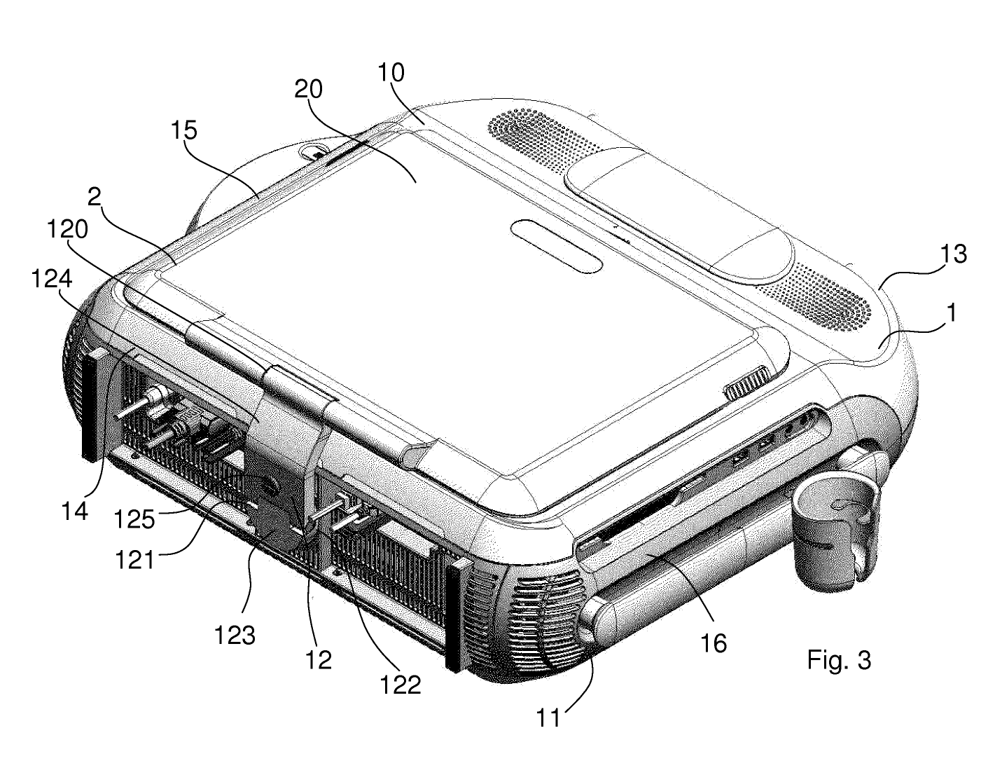

FIGS. 3 to 9 are the positions in which the monitor can be moved;

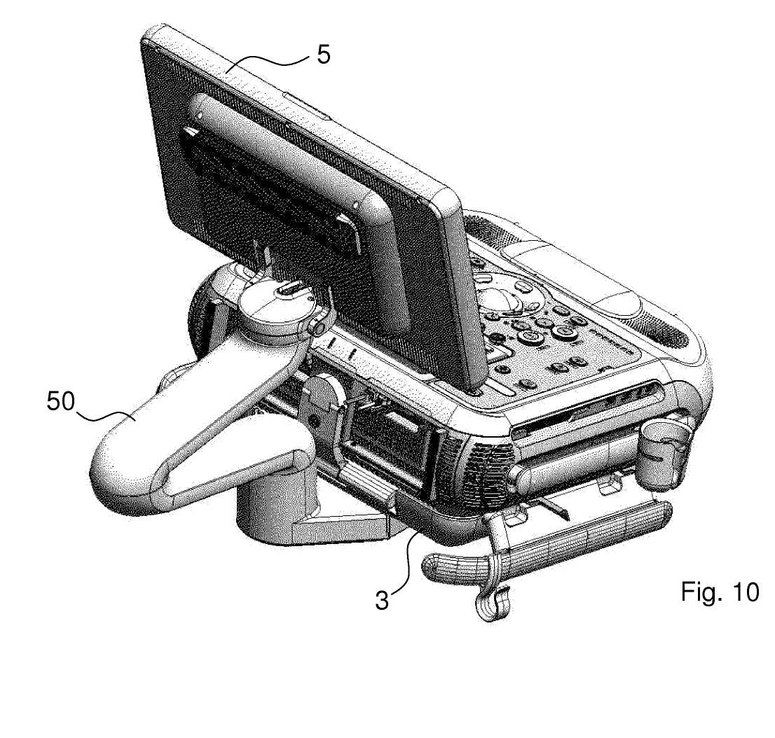

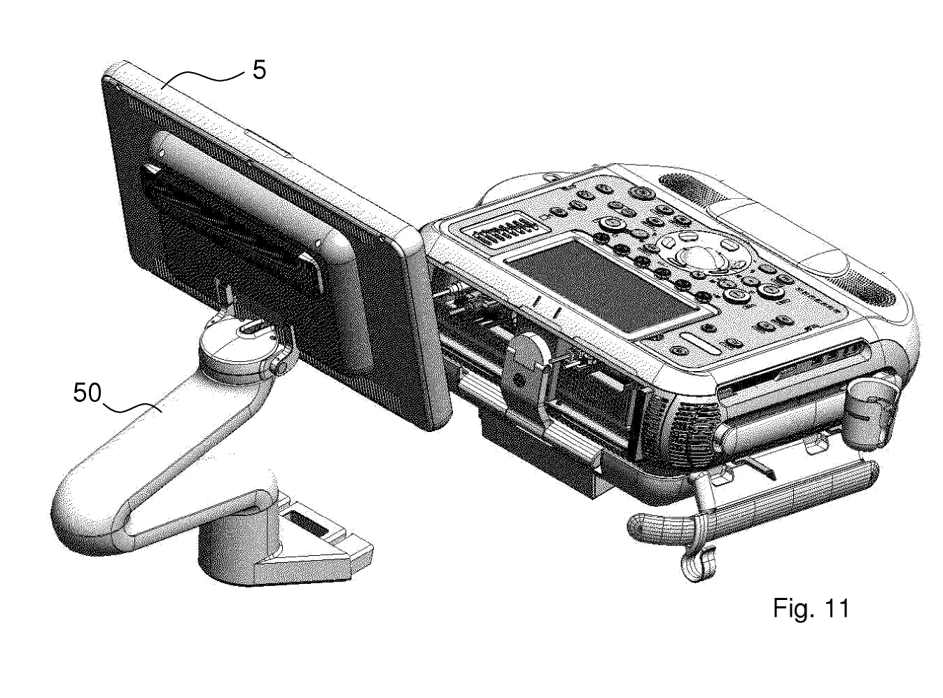

FIGS. 10 and 11 are the additional monitor coupled with the carriage and detached from the carriage respectively;

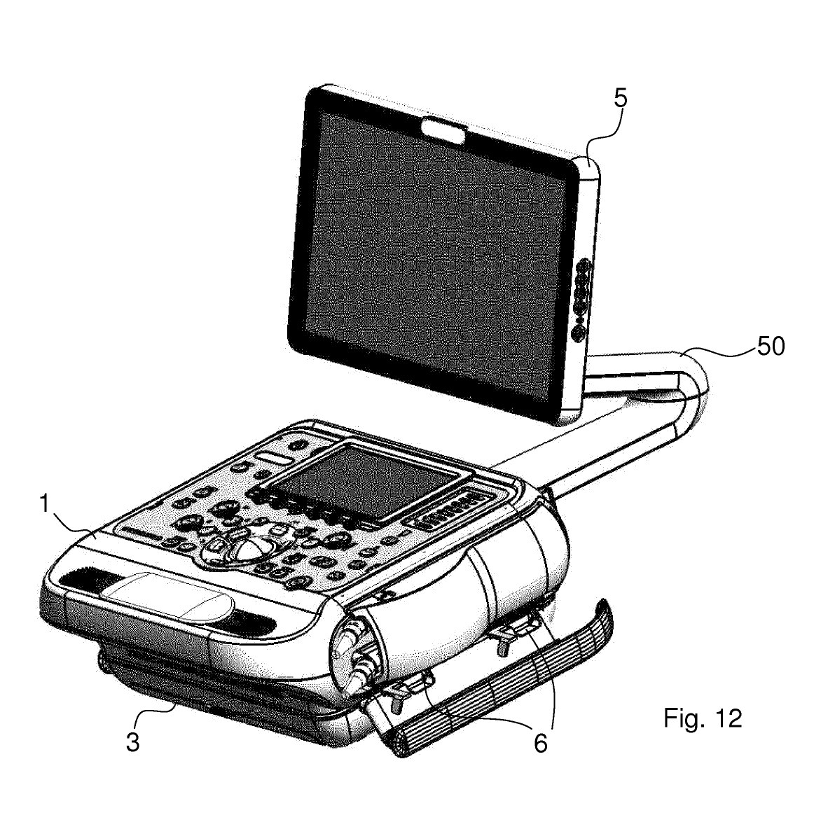

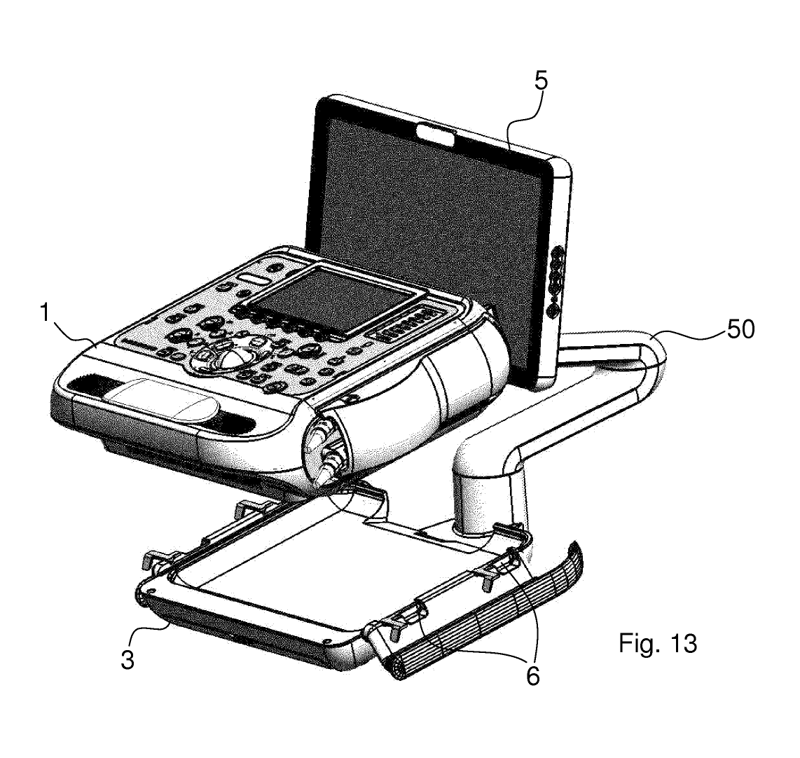

FIGS. 12 and 13 are the coupling of the machine to the carriage;

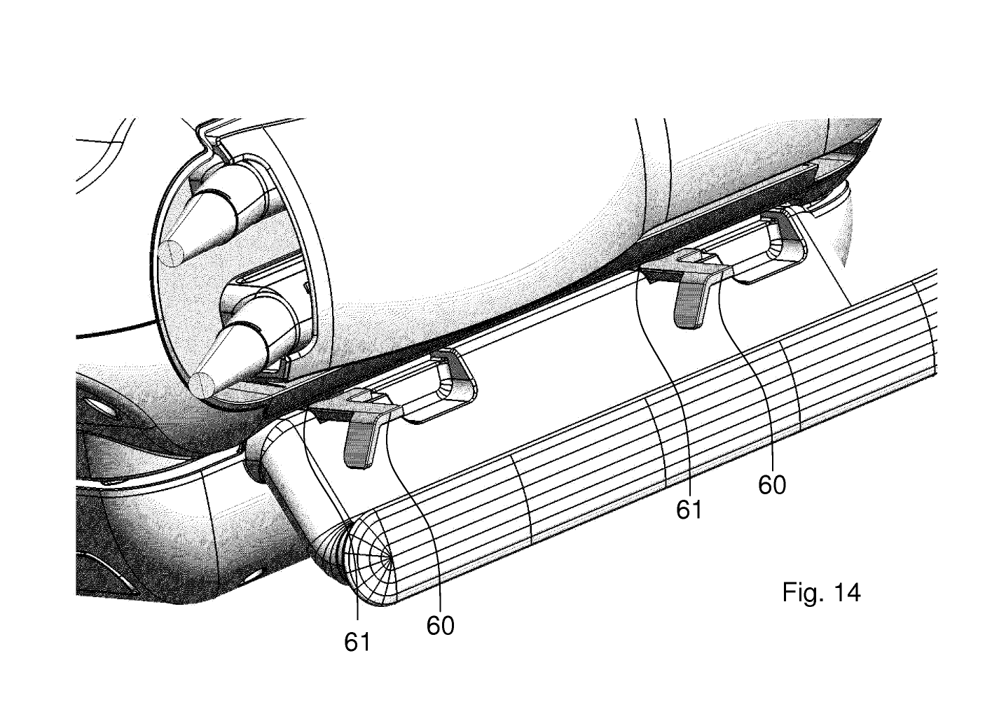

FIGS. 14 to 18 are a detail of the embodiment of the means for coupling the machine to the carriage;

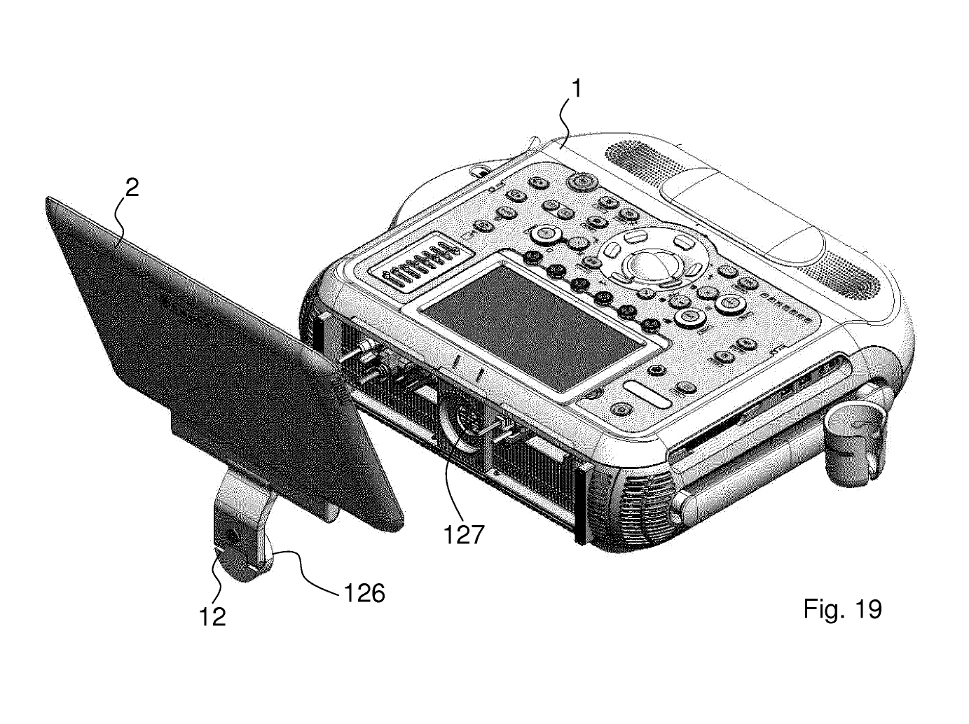

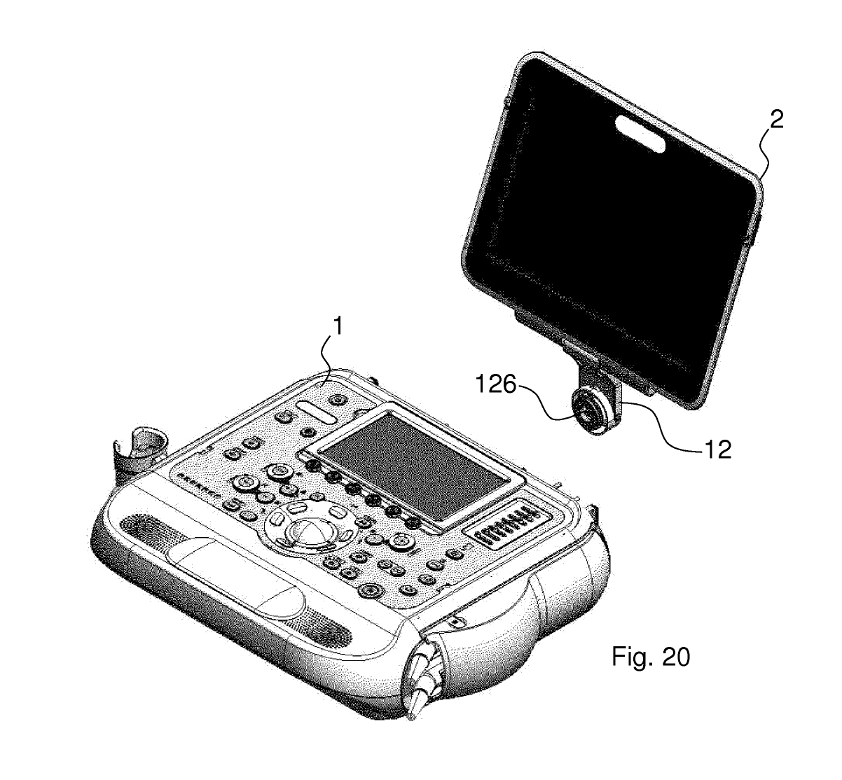

FIGS. 19 to 21 are the monitor detachable from the main body of the machine;

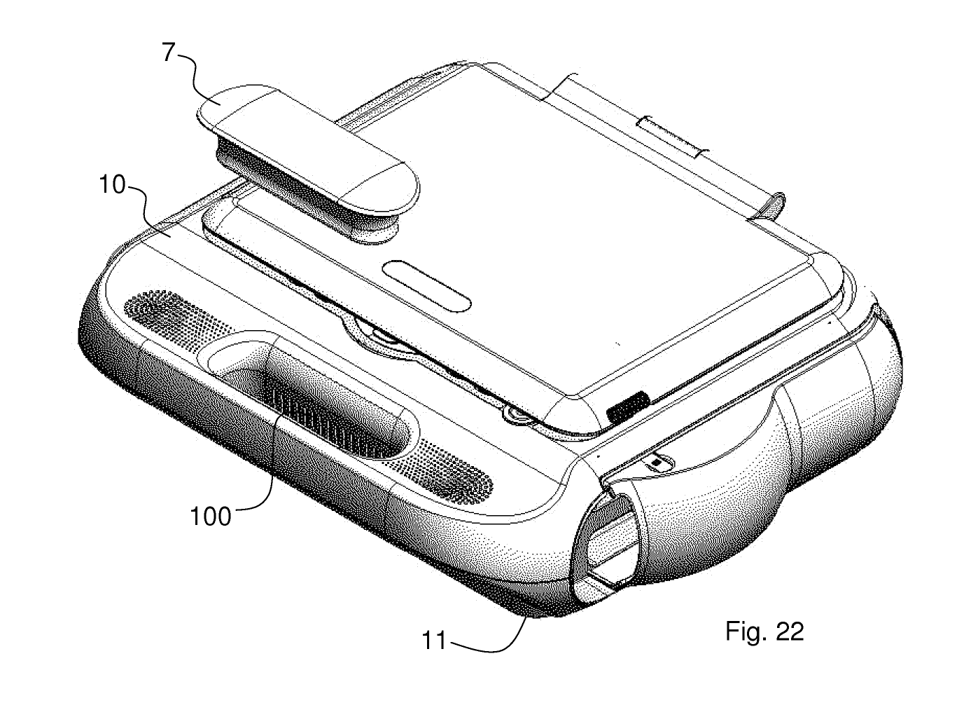

FIG. 22 is the handle and the wrist rest plug in the detached condition;

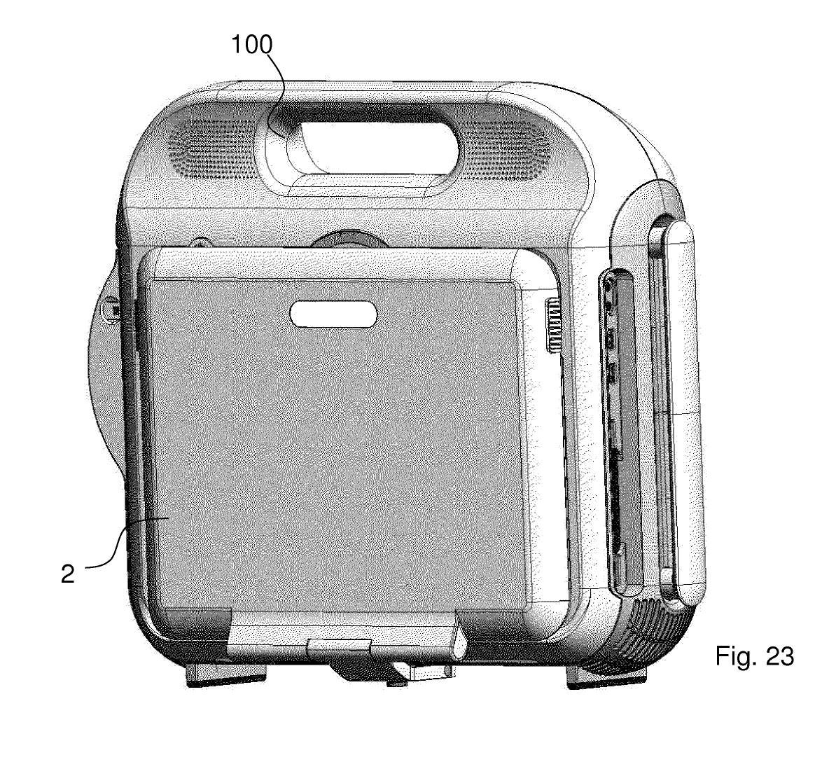

FIGS. 23 is the portable machine in the transport condition;

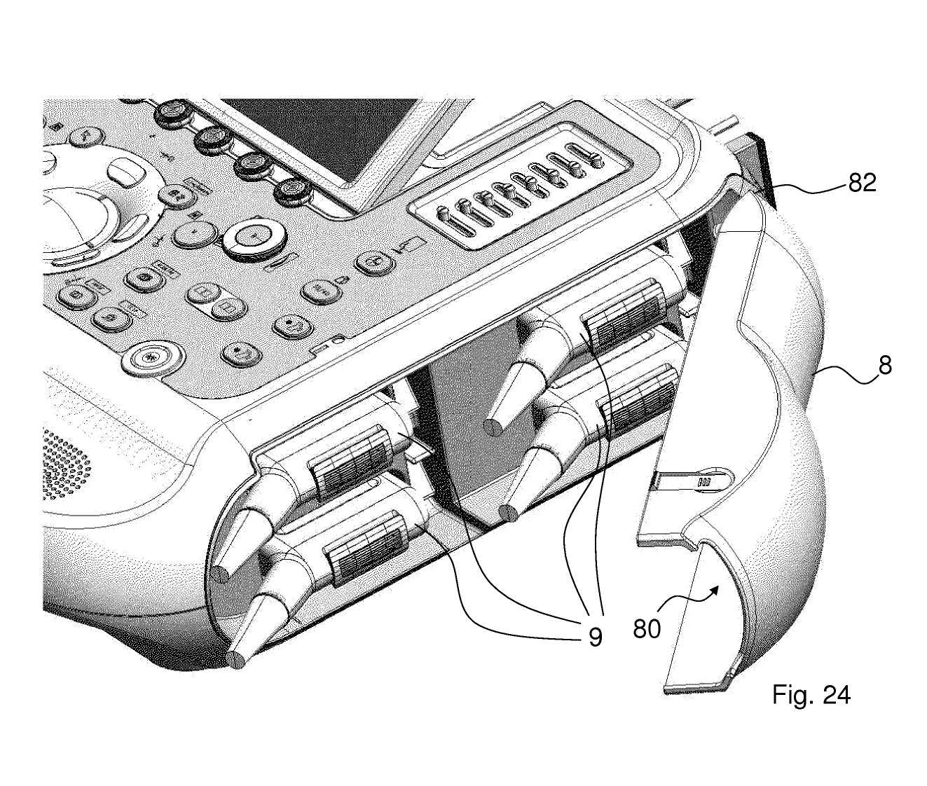

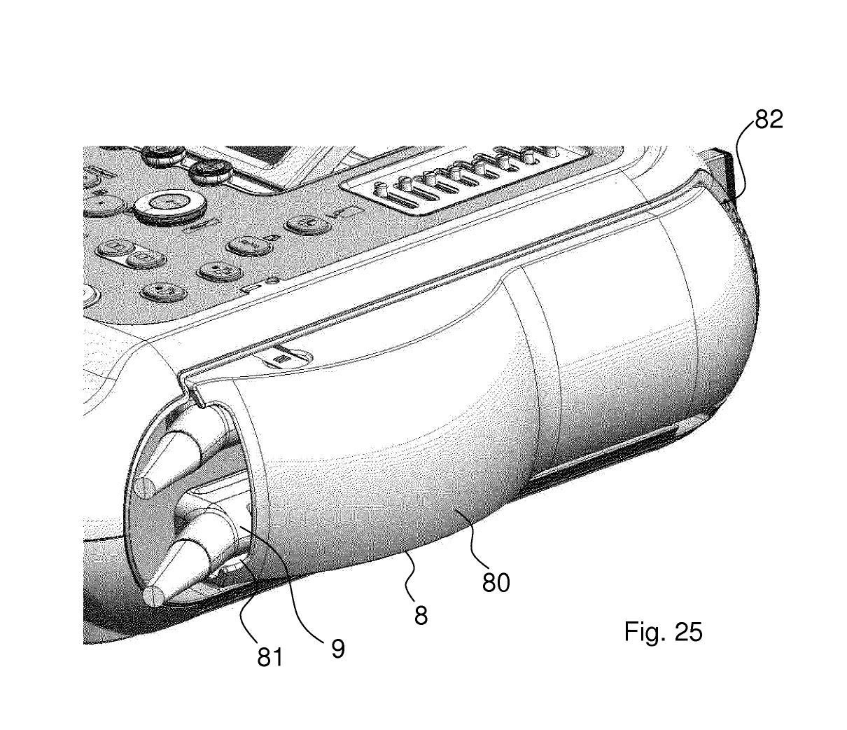

FIGS. 24 and 25 are one embodiment of the case in the opening and closing positions respectively;

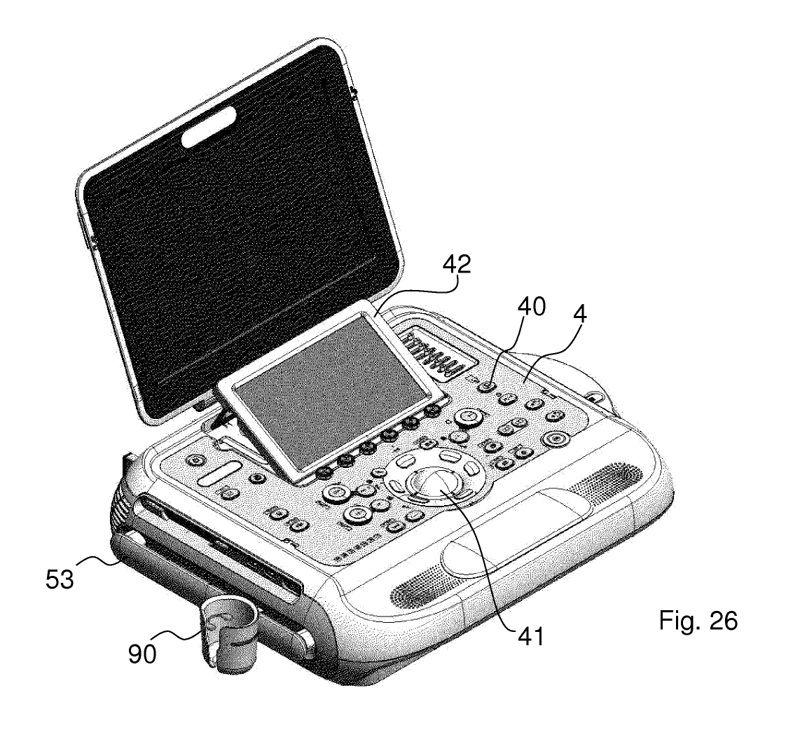

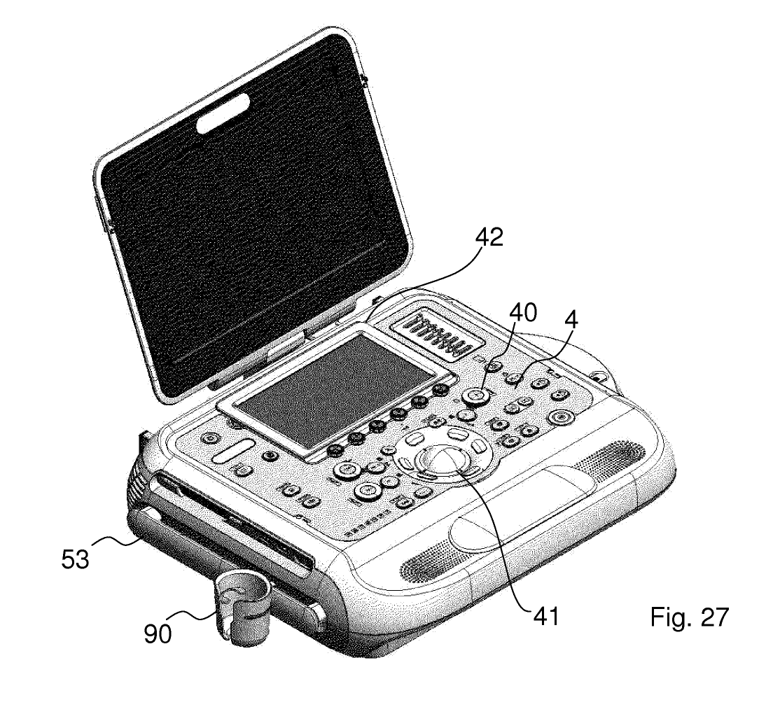

FIGS. 26 and 27 are a detail of one embodiment of the control interface, with a touch screen raised and not raised respectively.

FIG. 1 shows one non-limiting embodiment of the portable ultrasound machine of the present invention.

Such machine comprises a main body 1 having an upper surface 10 provided with a control user interface and a lower surface 11 resting on a supporting structure, preferably a carriage 3.

It is possible to provide supporting structures different than a conventional carriage for biomedical devices, such as for example fixed structures or the like.

As it can be seen in FIG. 2, the supporting structure 3 is provided with a housing seat 31 for the main body 1 of the portable machine, which housing seat 31 enters in contact with the lower surface 11 of the main body 1 when the portable machine is in the condition coupled with the supporting structure 3.

A communication interface is provided between the portable machine and the supporting structure, for example for connecting a processing unit inside the portable machine to an extension processing unit provided into the supporting structure 3.

It is possible to provide for such communication interface contacts of the so called docking station type, such that the connection is automatically established upon resting the machine into the housing seat 31.

The portable machine, such as shown for example in FIG. 3, has a substantially parallelepiped shape, delimited by said upper surface 10 and by said lower surface 11, which connect to each other by means of side walls, particularly a front wall 13, a rear wall 14, a first side wall 15 and a second side wall 16.

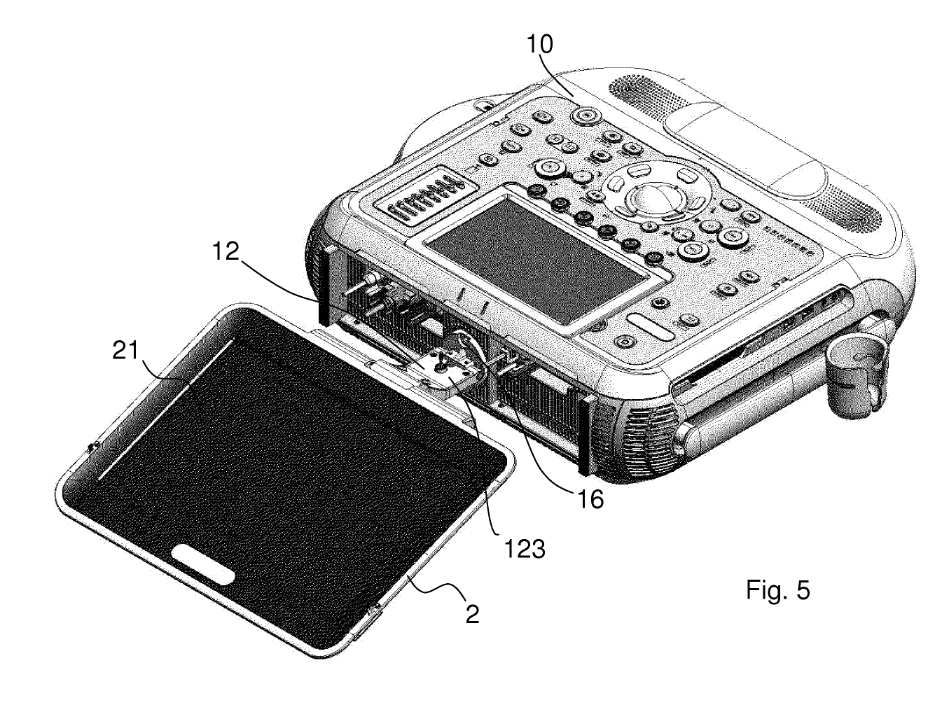

The portable machine comprises a monitor 2, provided with a front emitting side 21, for example seen in FIG. 5, a rear casing side 22 opposite to the front emitting side 21.

The monitor 2 is connected to the main body 1 by means of a joint 12 articulatable such that it can be moved in several positions such as shown in FIGS. 3 to 9.

The joint 12 is composed of two portions hinged to each other by a medial hinge 121, the first portion 123 of which is connected to a terminal 122 rotationally coupling with the main body 1, and a second portion connected to the monitor 2 by means of an end hinge 120.

The medial hinge 121 and the end hinge 120 have axes of rotation parallel to each other, while the rotational coupling terminal 122 has an axis of rotation perpendicular to the other two.

The rotational coupling terminal 122 is connected to the rear wall 14 at a point equidistant from the upper wall 10 and from the lower wall 11.

FIG. 3 shows the monitor 2 in the first position, where the monitor 2 is arranged parallel to the upper surface 10, with the front emitting side 21 facing towards the upper surface 10.

In such first position the monitor 2 is folded on the main body 1 and it covers the control interface 4.

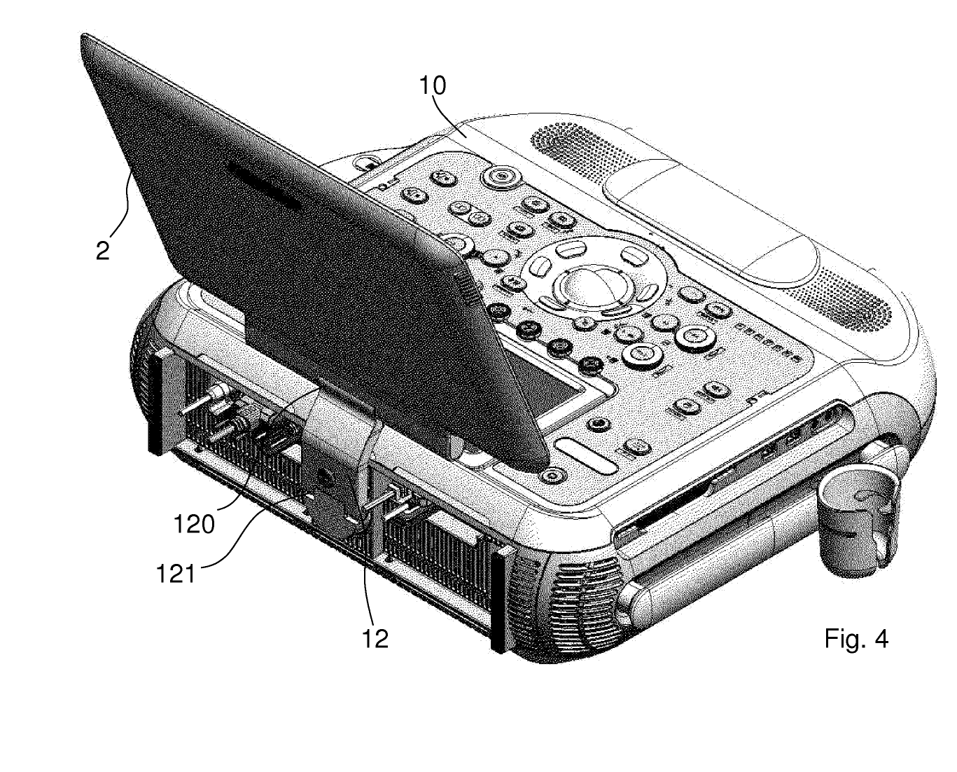

From such first position the monitor 2 can be moved merely by rotating it about the end hinge 120 to a second position, shown in FIG. 4, where it is arranged substantially vertical above the upper surface 10, such to allow the user to display the acquired ultrasound images.

There is provided a screw 125 for tightening the joint 12 in position, which is engaged into a through hole obtained in the second portion 124, seen for example in FIGS. 3 and 4, and intended to cooperate with a corresponding threaded seat 126 provided in the first portion 123, seen for example in FIG. 5.

When the medial hinge 121 is bent at 90°, the tightening screw 125 can be engaged into the threaded seat 126, such to allow the monitor 2 only to rotate about the end hinge 120.

When the tightening screw 125 is unloosed or removed, the joint 12 can rotate about the medial hinge 121, allowing the monitor 2 to be placed for example in the position of FIG. 5, where the monitor 2 and the joint 12 are substantially on the same plane parallel to the upper surface 10, with the front emitting side 21 of the screen 2 faced upwards.

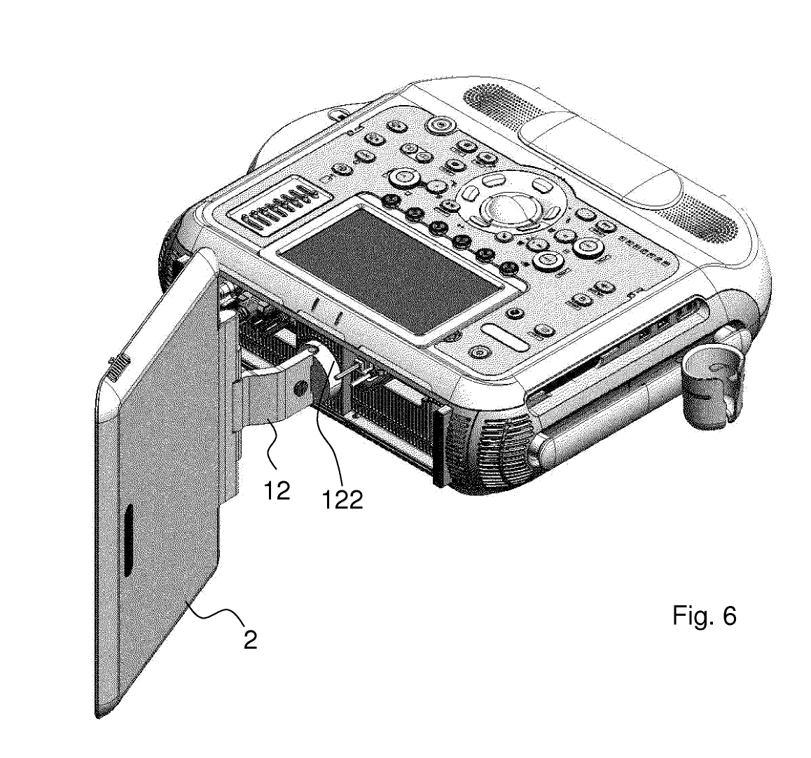

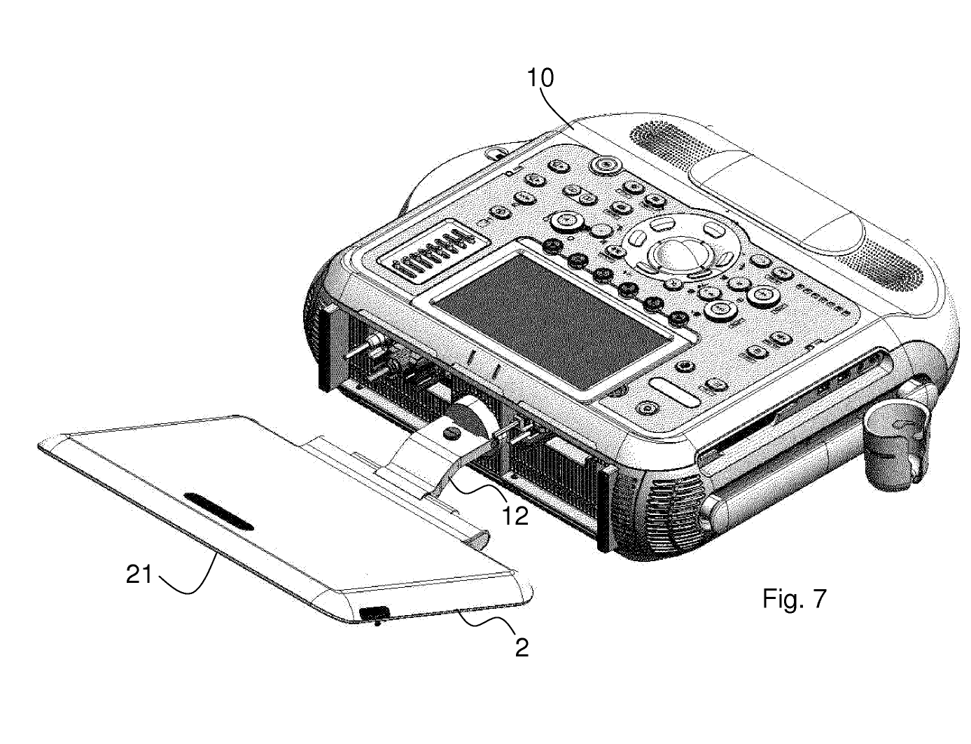

Now it is possible to rotate the joint 12 and the monitor 2 about the axis of rotation of the rotational coupling terminal 122, such as shown in FIG. 6, up to a position wherein the monitor and the joint 12 are substantially on the same plane parallel to the upper surface 10, but with the front emitting side 21 of the monitor 2 faced downwards, such as shown in FIG. 7.

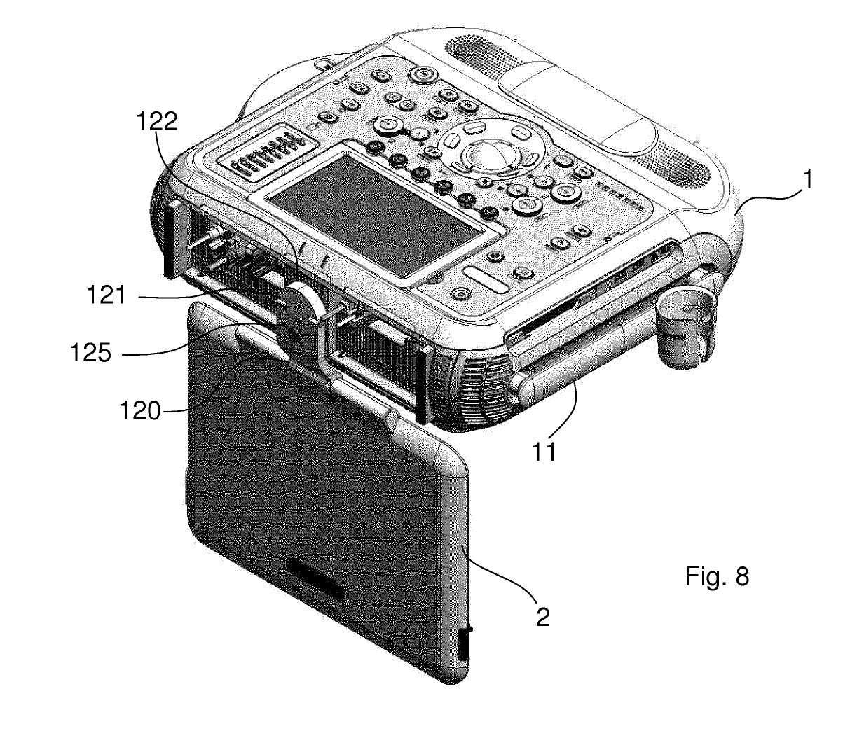

From such position, by keeping the rotational coupling terminal 122 fixed and by acting on the medial hinge 121, the monitor 2 is moved in a condition substantially symmetric to said second position with respect to the median horizontal plane passing by the main body 1, that is wherein it is placed substantially vertical underneath the lower surface 11, such as shown in FIG. 8.

In such position the tightening screw 125 is again screwed into the threaded seat 126, allowing only the end hinge 120 to rotate.

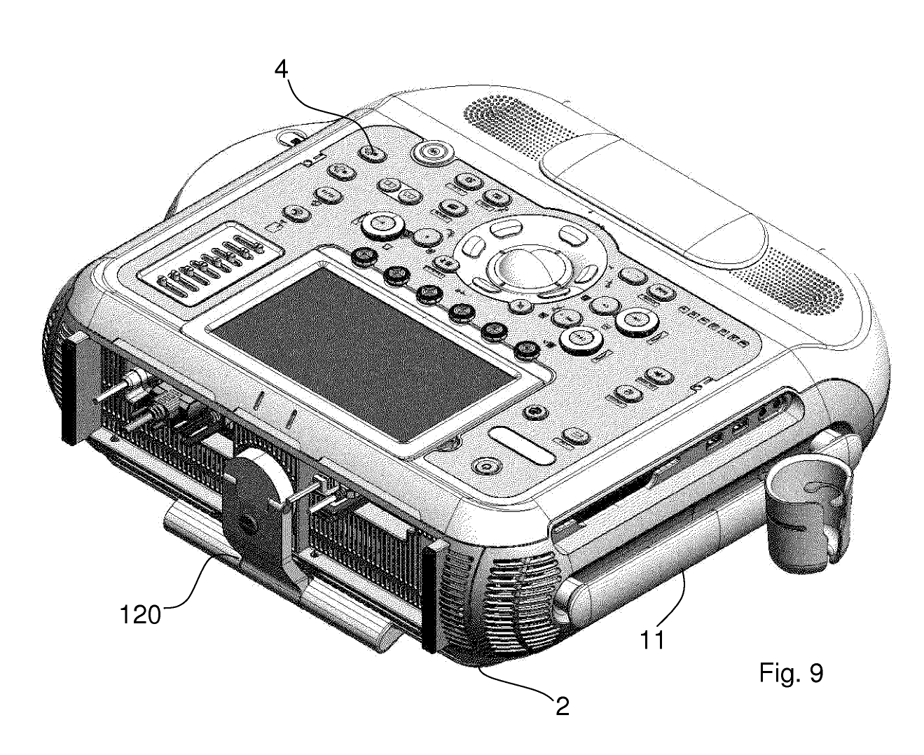

Thus the monitor 2 can be rotated about the end hinge 120 up to a third position, shown in FIG. 9, where it is arranged parallel to the lower surface 11, with the front emitting side 21 facing towards the lower surface 11.

In the third position the monitor 2 is folded against the lower surface 11, and therefore it is again in the non-operating position as in the first position shown in FIG. 3. But unlike what occurs in the first position, in the third position the monitor 2 does not cover the control interface 4, which therefore can be used by the user.

The monitor 2 is provided with a conventional coupling system in the closing position, such coupling system being provided on the main body both on the upper surface 10 and on the lower surface 11.

The supporting structure is provided with an additional monitor 5, said communication interface between the portable machine and the supporting structure being intended to display the images acquired by the portable machine on said additional monitor 5.

The additional monitor 5 is fastened to one end of an articulatable arm 50, which is removably connectable at the opposite end to the supporting structure 3.

FIGS. 10 and 11 show the additional monitor 5 in the condition coupled and detached from the supporting structure 3 respectively.

As an alternative it is possible to provide an additional monitor 5 mounted in a fixed manner on an articulatable arm 50 coupled to the supporting structure 3, or an additional monitor 5 removably mountable to an articulatable arm 50 mounted in a fixed manner on the supporting structure 3.

FIGS. 12 and 13 show the system for coupling the main body 1 to the housing seat 31 of the supporting structure 3.

It is possible to couple the portable machine to the supporting structure 3 with the monitor 2 in the second position, that is arranged vertical above the upper surface 10, and contemporaneously not to couple the additional monitor 5.

Thus the portable machine is supported by the supporting structure 3 but it uses the monitor 2.

As an alternative it is possible to couple the portable machine to the supporting structure 3 with the monitor 2 in the third position, that is folded and concealed underneath the ultrasound machine, and to couple to the supporting structure the additional monitor 5, upon which the images are displayed, such as shown for example in FIGS. 12 and 13.

Then it is possible to couple the portable machine to the supporting structure 3 with the monitor 2 in the second position and to couple the additional monitor 5, such to have two contemporaneously operating monitors.

There are provided means 6 for coupling the main body 1 to the supporting structure 3, which are placed on the side walls 15 and 16 and are spaced apart from each other at an extent slightly greater than the dimensions of the monitor 2, such that when said monitor 2 is moved in the third position it is interposed between said coupling means 6.

Said coupling means 6 are composed of levers 60 rotatable about fulcrums provided on the supporting structure 3 such to engage with corresponding slits 61 obtained in the side walls 14 and 15 of the main body 1.

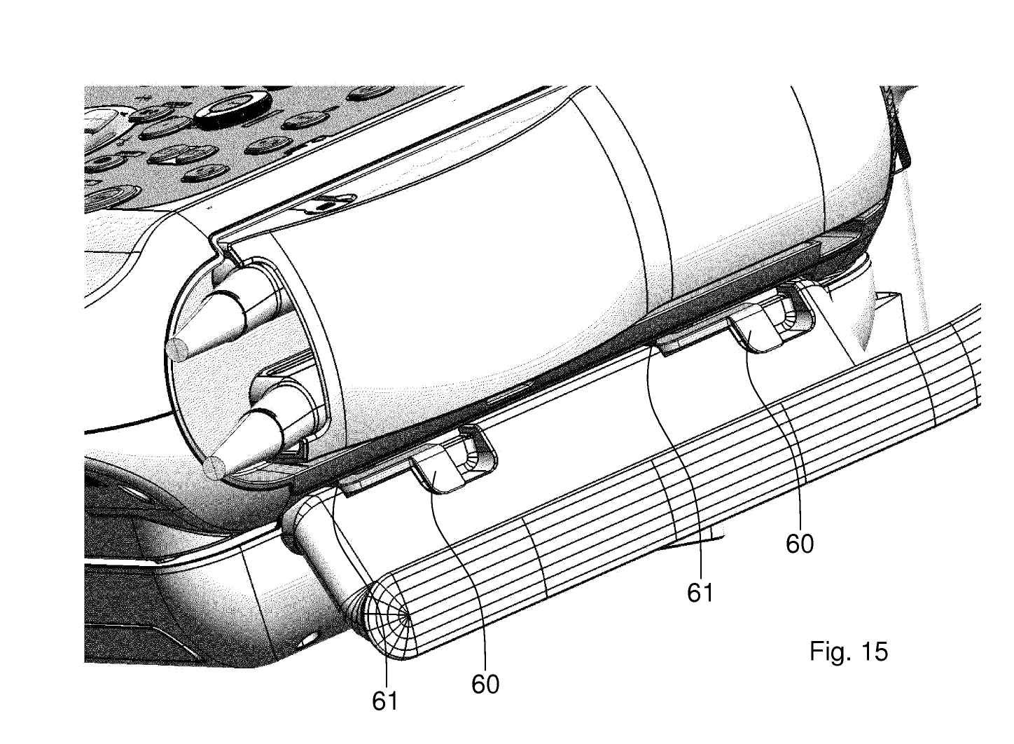

In FIG. 14 the levers 60 are shown in the opening position, wherein they are not engaged into the slits 61, while in FIG. 15 they are shown in the closing position.

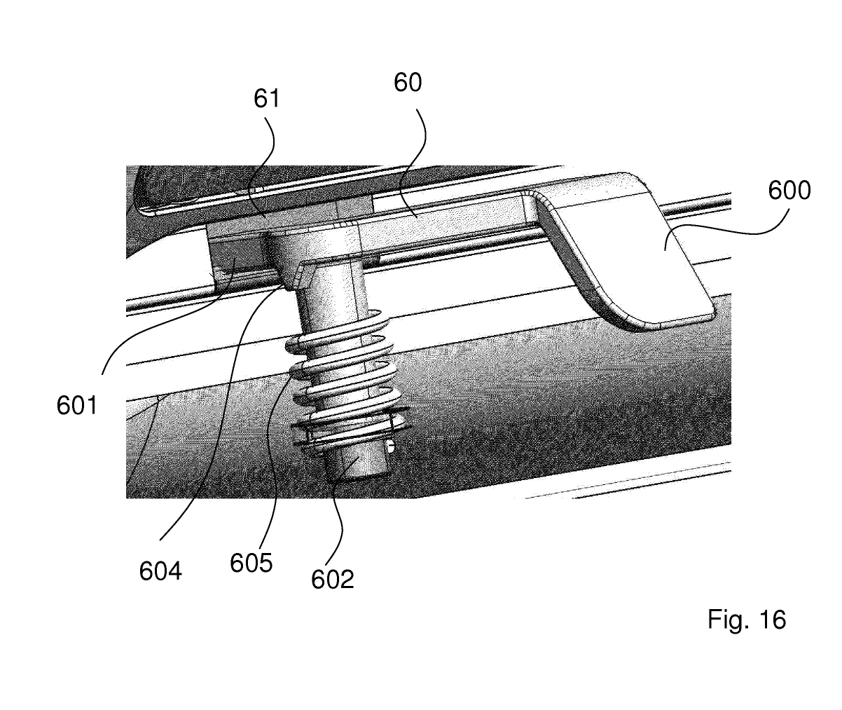

FIG. 16 shows a detail of one lever 60, which has a driving end 600, an engagement end 601 and one fulcrum 602.

There is further provided a retaining spring 605 that keeps the lever 60 urged downwards, the lever 60 being provided with a locking tooth 64 that projects downwardly and which is intended to engage in two corresponding seats 610 and 611 for the opening position and the closing position respectively.

The retaining spring 605 guarantees the lever to remain firmly in one of the two positions unless the user exerts voluntarily an opposing action that allows the locking tooth 64 to go out from the seat 610 or 611, thus guaranteeing the lever 60 to pivot about the fulcrum 602.

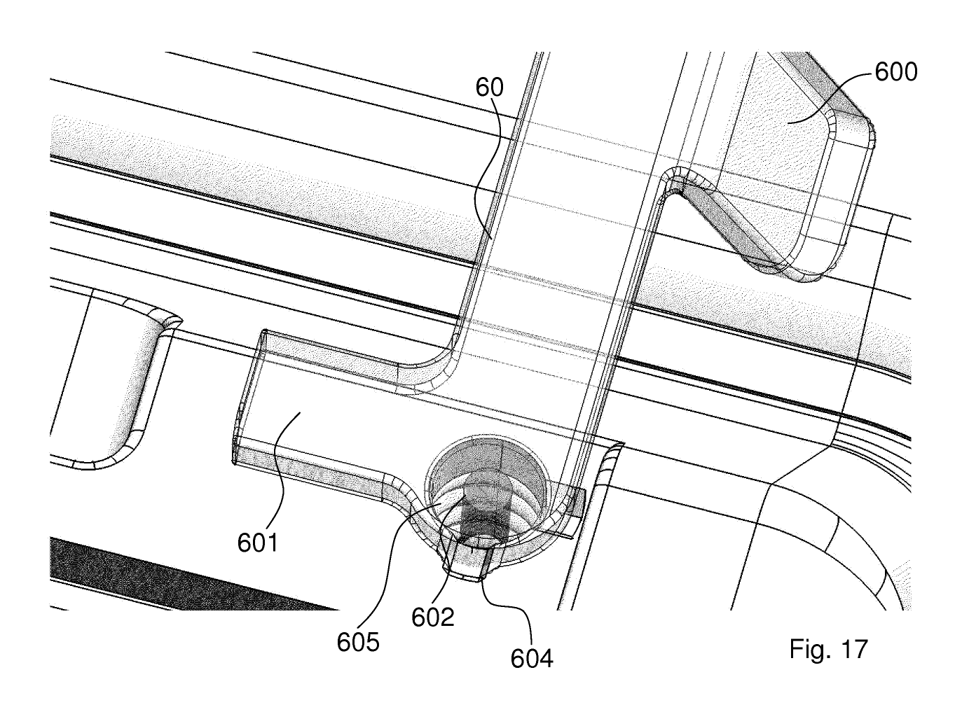

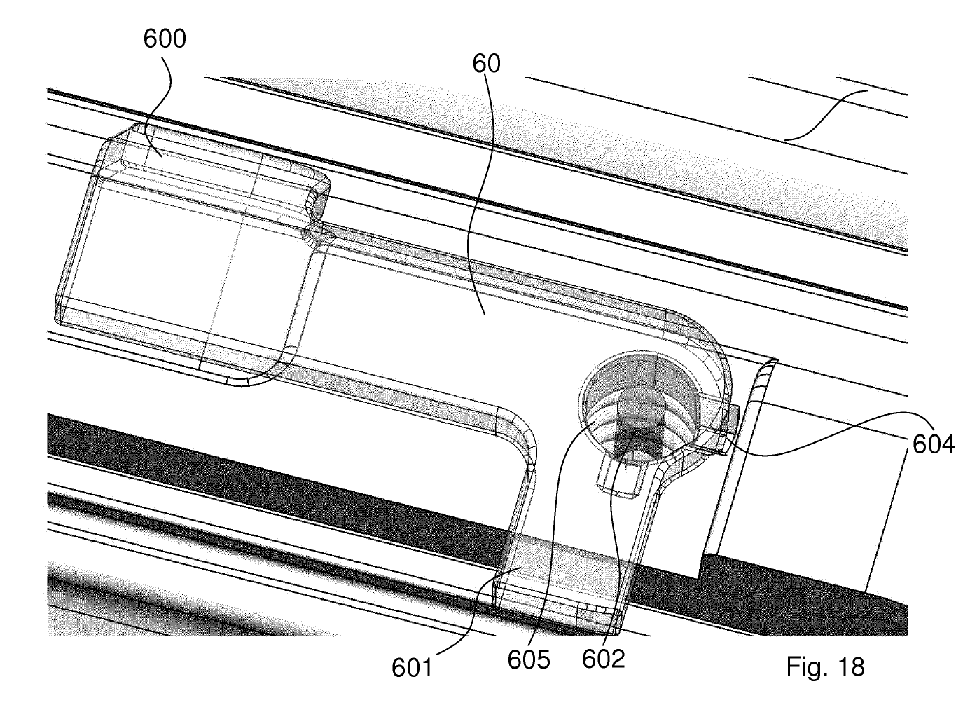

FIGS. 17 and 18 show the lever 60 in the opening position and in the closing position respectively. FIGS. 18 and 19 show the monitor 2 detachable from the main body 1 it being mechanically and electrically connectable to the supporting structure 3, preferably to the end of the articulable arm 50, or at an intermediate point of the arm 50 or directly on the supporting structure 3.

The joint 12 is provided with a coupling terminal 126 intended to be removably engaged with a corresponding coupling terminal 127 provided on the main body 1 or on the supporting structure 3, both the terminals 126 and 127 being provided with electrical contacts that are automatically connectable when said terminals 126 and 127 are connected to each other and that guarantee the electrical connections when rotating said joint 12.

According to one embodiment the coupling terminal provides a central pinion and snap coupling means, and it has rotating contact terminals.

There is provided a first stationary plate that bears the contacts, which are rotatably connected to tracks or other conductors provided in the monitor 2 and such plate is mounted at the end of an attachment pinion which is rotatable about its own axis.

The coupling terminals 126 and 127 can be composed of a hollow cylindrical element and of an element composed of a cylindrical external wall with a central pin respectively, such to guarantee a shape fit.

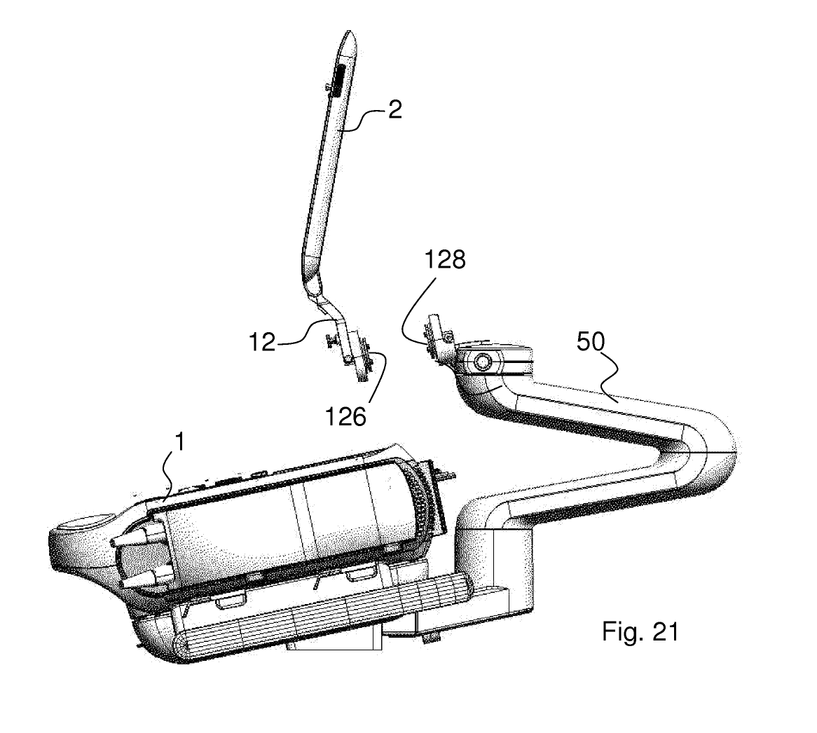

Such as shown in FIG. 21 a coupling terminal 128, completely similar to the terminal 127, is provided at the end of the articulatable arm 50, such to couple the monitor 2 to the supporting structure 3, once it is removed from the main body 1 of the portable machine.

FIG. 22 shows a transport handle composed of a recess 100 obtained on the upper surface 10.

In the example in the figure the recess 100 is a through recess and it puts the upper surface 10 in communication with the lower surface 11, thus forming an handle that can be more easily grasped by the user.

The mouth of the recess 100 is coverable at least partially by a removable plug 7 made of an elastically pliable material, such as for example elastomer, with such a size that it acts as a wrist rest for the user.

The plug 7 can be provided only at the upper surface 10 or it can have a matching plug on the lower surface 11.

FIG. 23 shows the portable ultrasound machine in the transport condition, with the transport handle faced upwardly and with the monitor 2 closed in the first position.

In the side wall 15 there are provided four connectors 9 for the same number of ultrasound probes, a protecting case 8 for the connectors 9 being provided, shown in FIGS. 24 and 25.

The case 8 is connected to the side surface 15 such that it is movable from an opening position to a closing position and it is shaped in such a manner to form in the closing position a space 80 housing the cables of the probes and to form an outlet opening 81 for the cables.

One end of the case 8 is rotatably coupled to the side surface 15 by a hinge 82 such that the case 8 is rotatable from the opening position to the closing position where it adheres against the side wall 15, the opening 81 being at one end of the case 8 opposite to the hinge 82.

The case 8 has a concave shape and it is provided with different depths such that in a direction opposite to the side wall 15 it has a widening at the connectors and a narrowing at the opening 81, it being made rounded with a predetermined radius of curvature such to form an external convex side and an internal concave side and the curvature being such that the maximum distance from the side wall 15 is obtained at the connectors while such distance decreases in the direction of the hinge 82 and in the direction of the opening 81.

Such widening allows the cables of the connectors 9 that are nearest to the hinge 82 to have the space necessary for passing the connectors 9 that are nearest to the opening 81.

At the opening 81 on the contrary the case 8 narrows again for reducing the external overall dimensions and for guaranteeing only the space necessary for allowing the cables to exit. The case 8 can be made as sliding, instead of opening like a compass.

The control user interface 4 comprises a plurality of buttons 40, a trackball 41, and a touch screen 42 that can be rotated from a deployed position, shown in FIG. 26, to a retracted position, shown in FIG. 27.

Moreover a supporting and translating bar 53 for glass-like elements 90 supporting the ultrasound probes is coupled to the side wall 16.

The invention claimed is:

1. A portable ultrasound machine, configured to be removably coupled to a supporting structure, (3), comprising:
a main body (1), and
a monitor (2) connected to the main body (1) by a joint (12),
wherein the main body (1) has an upper surface (10) provided with a control user interface (4), a lower surface (11) resting in a housing seat (31) of said supporting structure (3), and one or more side walls (13, 14, 15, 16) connecting said upper surface (10) to said lower surface (11),
wherein said monitor (2) is provided with a front emitting side (21) and a rear casing side (20) opposite to said front emitting side (21), and there being provided a communication interface between said main body and said supporting structure (3),
wherein said joint (12) is configured to be articulated such that said monitor (2) is alternately movable between a first position, wherein said monitor is disposed parallel to said upper surface (10), with the front emitting side (21) facing towards said upper surface (10), and a second position, wherein said monitor is arranged substantially vertical above said upper surface (10), and
wherein said joint (12) is further configured to be articulated such that said monitor (12) is alternately movable to a third position, wherein said monitor is arranged parallel to said lower surface (11), with the front emitting side (21) faced towards said lower surface (11).

2. The portable ultrasound machine according to claim 1, wherein said joint (12) has at least two pivot points (120, 122), axes of rotation of at least two of said pivot points (120, 122) being perpendicular to each other.

3. The portable ultrasound machine according to claim 1, wherein said joint (12) has at least three pivot points (120, 121, 122), axes of rotation of at least a first and at least a second pivot points (120, 121) being parallel to each other and perpendicular to an axis of rotation of at least a third pivot point (122).

4. The portable ultrasound machine according to claim 1, wherein said main body (1) has a parallelepiped shape, such that said side walls comprise a front wall (13), a rear wall (14), and two side walls (15, 16), said joint (12) being connected to said rear wall (14) at a point equidistant from said upper surface (10) and from said lower surface (11).

5. The portable ultrasound machine according to claim 1, further comprising (6) members configured to couple the machine to said supporting structure (3), said members (6) being provided on said lower surface (11) or on said side walls (15, 16), on sides opposite to each other of said lower surface (10), said members being spaced from each other at an extent corresponding to or higher than outer dimensions of said monitor (2), such that when said monitor (2) is moved in said third position it is interposed between said members (6).

6. The portable ultrasound machine according to claim 1, wherein said supporting structure (3) is provided with at least an additional monitor (5), said communication interface between said machine and said supporting structure (3) being configured to enable a display of images acquired by said machine on said additional monitor (5).

7. The portable ultrasound machine according to claim 6, wherein said additional monitor (5) is removably connectable to said supporting structure (3), or is connected or connectable to an arm (50) removably connectable to said supporting structure (3).

8. The portable ultrasound machine according to claim 1, wherein said monitor (2) is detachable from said machine and is mechanically and electrically connectable to said supporting structure (3).

9. The portable ultrasound machine according to claim 8, wherein said joint (12) is provided with a coupling terminal (126) configured to be removably engaged with a corresponding coupling terminal (127) provided on said main body (1) or on said supporting structure (3), the terminals (126, 127) of said joint and on said main body being provided with electrical contacts that are automatically connected when said terminals (126, 127) are coupled one to the other, thereby providing and electrical connections during a rotation of said joint (12).

10. The portable ultrasound machine according to claim 1, wherein said communication interface comprises docking station contacts, such that a connection is automatically established upon placement of said machine in said housing seat (31).

11. The portable ultrasound machine according to claim 1, further comprising a transport handle defined by a recess (100) formed on said upper surface (10), a mouth of said recess (100) being at least partially coverable by a removable plug (7) made of an elastically pliable material sized to provide a wrist rest for a user.

12. The portable ultrasound machine according to claim 1, wherein in one side wall (15) there is provided at least one connector (9) for at least one ultrasound probe, further comprising a case (8) protecting said at least one connector (9), said case (8) being connected to said side wall (15) such that said case is movable from an opening position to a closing position and is shaped to form in the closing position a housing space (80) for at least a cable of said at least one probe and to form an outlet opening (81) for said cable.

13. The portable ultrasound machine according to claim 12, wherein an end of said case (8) is hinged to said side wall (15) such that said case (8) is rotatable from said opening position to said closing position in which said case is in contact with said side wall (15), said case (8) having a concave shape and a depth such that in a direction opposite to said side wall, said case has a widening at said at least one connector (9) and a narrowing at said outlet opening (81) for said cable.

14. A supporting carriage for a portable ultrasound machine comprising:
removable members configured to fasten (6) said portable ultrasound machine to a support structure; and
at least one terminal provided with a connector for removable mechanical and electrical coupling of a monitor (2).

15. The supporting carriage according to claim 14, wherein said ultrasound machine comprises:
a main body (1), and
a monitor (2) connected to the main body (1) by a joint (12),
wherein the main body (1) has an upper surface (10) provided with a control user interface (4), a lower surface (11) resting in a housing seat (31) of said supporting carriage (3), and one or more side walls (13, 14, 15, 16) connecting said upper surface (10) to said lower surface (11),
wherein said monitor (2) is provided with a front emitting side (21) and a rear casing side (20) opposite to said front emitting side (21), and there being provided a communication interface between said main body and said supporting carriage (3),
wherein said joint (12) is configured to be articulated such that said monitor (2) is alternately movable between a first position, wherein said monitor is disposed parallel to said upper surface (10), with the front emitting side (21) facing towards said upper surface (10), and a second position, wherein said monitor is arranged substantially vertical above said upper surface (10), and
wherein said joint (12) is further configured to be articulated such that said monitor (12) is alternately movable to a third position, wherein said monitor is arranged parallel to said lower surface (11), with the front emitting side (21) faced towards said lower surface (11).
